(19) United States
(12) Patent Application Publication
(10) Pub. No.: US 2020/0082931 A1
(43) Pub. Date: Mar. 12, 2020

(54) DIAGNOSTIC SUPPORT APPARATUS

(71) Applicant: FUJIFILM Corporation, Tokyo (JP)

(72) Inventors: Haruyasu NAKATSUGAWA, Tokyo (JP); Tsuyoshi HIRAKAWA, Tokyo (JP); Hiroshi HIRAMATSU, Tokyo (JP)

(73) Assignee: FUJIFILM Corporation, Tokyo (JP)

(21) Appl. No.: 16/564,245

(22) Filed: Sep. 9, 2019

(30) Foreign Application Priority Data
Sep. 11, 2018 (JP) 2018-169818

(51) Int. Cl. G16H 30/40; G06K 9/62; G16H 50/20; G16H 15/00

(52) U.S. Cl. CPC G16H 30/40; G16H 15/00; G16H 50/20; G06K 9/6215

(57) ABSTRACT

In a case where a target image that is a diagnostic target is selected and a reference image reference tag is operated, a similar image similar to the target image is detected. Then, an image referred to in the past diagnosis using the similar image is detected, and the detected image is displayed on a diagnostic support screen as a support image. On the diagnostic support screen, the support image is displayed as a thumbnail image. In a case where any thumbnail image is selected, the selected support image is enlarged and displayed. On the diagnostic support screen, the degree of effectiveness indicating how much the support image has been referred to is displayed so as to be associated with the support image.

### CROSS-REFERENCE TO RELATED APPLICATIONS

This application claims priority under 35 U.S.C § 119(a) to Japanese Patent Application No. 2018-169818 filed on Sep. 11, 2018. The above application is hereby expressly incorporated by reference, in its entirety, into the present application.

### BACKGROUND OF THE INVENTION

### 1. Field of the Invention

The present invention relates to a diagnostic support apparatus.

### 2. Description of the Related Art

It is widely performed in medical facilities that a doctor or the like makes a diagnosis using a medical image obtained by examining (imaging) a patient. In the case of making a diagnosis, it is preferable to take past cases into consideration. For this reason, the following JP5700964B discloses a configuration in which medical images with the same diagnostic purpose and/or the same diagnostic part as medical images that are current diagnostic targets, among past medical images, are displayed as reference images to be a reference for diagnosis.

### SUMMARY OF THE INVENTION

In the above JP5700964B, medical images with the same diagnostic purpose and/or the same diagnostic part are displayed. However, it was insufficient to take past cases into consideration without knowing the history of past diagnoses performed using the past medical images with the same diagnostic purpose and/or the same diagnostic part as described above (without knowing until the diagnosis was made focusing on which point in the history of the past diagnosis).

The invention has been made in view of the above background, and it is an object of the invention to provide a diagnostic support apparatus capable of performing a diagnosis by taking the past cases into consideration more reliably.

In order to solve the aforementioned problem, a diagnostic support apparatus of the invention is a diagnostic support apparatus for supporting a diagnosis performed using a medical image. The diagnostic support apparatus comprises: a correspondence information storage unit that stores correspondence information indicating a correspondence relationship between a medical image, which is a diagnostic target in a past diagnosis, and a medical image referred to in the past diagnosis; a similar image detection unit that sets a medical image, which is a current diagnostic target, as a target image and detects a similar image, which is similar to the target image, from medical images that are diagnostic targets in the past diagnosis; a reference image detection unit that detects a reference image, which is a medical image referred to in diagnosing the similar image, using the correspondence information; and a display controller that displays the reference image on a display unit.

A plurality of the reference images may be displayed as a list.

The reference image may be displayed on the display unit as a thumbnail image subjected to reduction processing.

A reference image storage unit that stores the reference image so as to be associated with patient information regarding a patient of a subject of the target image may be provided.

A patient information storage unit that stores patient information regarding a patient of a subject of the reference image so as to be associated with patient information regarding a patient of a subject of the target image may be provided.

The similar image detection unit may detect an image, which includes a region similar to a designated partial region of the target image, as the similar image.

The correspondence information may include a degree of effectiveness indicating a degree of influence on the past diagnosis, and the display controller may determine a display mode of the reference image using the degree of effectiveness.

The display controller may display the degree of effectiveness so as to be associated with the reference image.

The display controller may determine a display order of the reference image using the degree of effectiveness.

The correspondence information may include the number of references in the past diagnosis, and the display controller may determine a display mode of the reference image using the number of references.

The display controller may display the number of references so as to be associated with the reference image.

The display controller may determine a display order of the reference image using the number of references.

The reference image detection unit may detect an image referred to in a past diagnosis in which the reference image is a diagnostic target, as a secondary reference image, using the correspondence information, and the display controller may display the secondary reference image on the display unit.

The display controller may display the similar image on the display unit as the reference image.

According to the invention, since the reference image referred to (displayed) in the past diagnosis using a similar image similar to the medical image to be diagnosed is displayed, it is possible to perform a diagnosis by taking the past cases into consideration more reliably.

### BRIEF DESCRIPTION OF THE DRAWINGS

FIG. 1 is a schematic diagram of a medical information system.

FIG. 2 is an explanatory diagram of an interpretation report.

FIG. 3 is an explanatory diagram of an interpretation DB server.

FIG. 4 is an explanatory diagram of each processing unit constructed in an interpretation DB server.

FIG. 5 is an explanatory diagram of a diagnostic support screen.

FIG. 6 is an explanatory diagram showing how a support image is detected.

FIG. 7 is an explanatory diagram of a diagnostic support screen.

FIG. 8 is an explanatory diagram of a diagnostic support screen.

FIG. 9 is an explanatory diagram of an enlarged screen displayed in a case where a support image is referred to.

FIG. 10 is an explanatory diagram of a diagnostic support screen.

### DESCRIPTION OF THE PREFERRED EMBODIMENTS

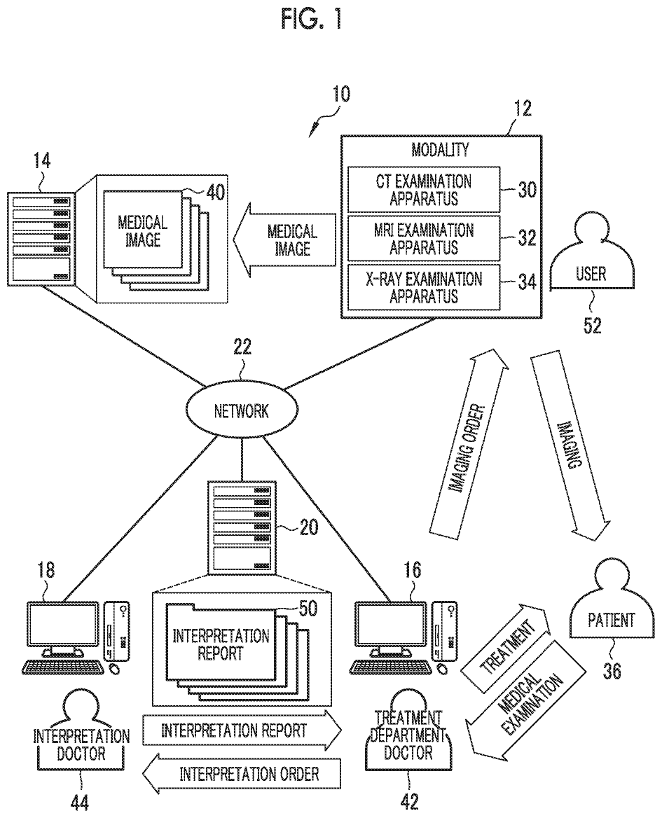

In FIG. 1, a medical information system 10 is constructed in a medical facility, such as a hospital, and comprises a modality 12, a medical image DB server 14, a clinical terminal 16, an interpretation terminal 18, and an interpretation DB server 20 (diagnostic support apparatus). These are communicably connected to each other through a network 22, such as a local area network (LAN) constructed in the medical facility.

The modality 12 is an examination apparatus that acquires a medical image 40 by imaging a patient 36, such as a computed tomography (CT) examination apparatus 30, a magnetic resonance imaging (MRI) examination apparatus 32, and an X-ray examination apparatus 34. The medical image DB server 14 is a so-called picture archiving and communication system (PACS) server that stores and manages the medical image 40 obtained by examination (imaging) using the modality 12. The clinical terminal 16 and the interpretation terminal 18 are, for example, computers such as personal computers. The clinical terminal 16 is used in a case where a treatment department doctor (or a clinician) 42 who determines the treatment policy of the patient 36 or performs actual treatment creates an electronic medical record, inputs examination reservation information to be described later, and the like. The interpretation terminal 18 is used in a case where an interpretation doctor 44 who interprets the medical image 40 creates a new interpretation report 50 or verifies the created interpretation report 50. In the present embodiment, an example will be described in which interpretation performed by the interpretation doctor 44 corresponds to the diagnosis of the invention.

In addition to the above, the medical information system 10 comprises a hospital information system (HIS) and a radiology information system (RIS).

The HIS receives various kinds of information relevant to medical practices of the medical facility, such as electronic medical records, accounting information, examination reservation information, and medication prescription information, from each department of the medical facility, such as a treatment department and a radiology department, and stores and manages the various kinds of information. Patient information is registered in the electronic medical record. The patient information has, for example, items of a patient identification (ID) for identifying each patient 36 and the name, sex, date of birth, age, height, and weight of the patient 36. The examination reservation information is input by the treatment department doctor (or the clinician) 42 through the clinical terminal 16. The examination reservation information includes not only various examination reservations, such as a blood test and an endoscopic examination, but also an imaging order that is an examination reservation using the modality 12. The imaging order is for instructing a user 52 of the modality 12 to perform imaging.

The RIS receives the imaging order input through the clinical terminal 16, and stores and manages the imaging order. The imaging order has, for example, items of an order ID for identifying each order, a doctor ID of the treatment department doctor (or the clinician) 42 who inputs the imaging order, the type of the modality 12 used in the imaging order, a patient ID of the patient 36 to be imaged, and an imaging part or direction. The imaging part includes not only rough parts in a case where the human body is roughly divided, such as the head, the chest, and the abdomen, but also parts obtained by subdividing the parts. The direction includes supine, prone, recumbent, and the like.

The RIS transmits the imaging order to the modality 12. For example, the user 52 of the modality 12 checks the imaging order on the display of the console of the modality 12, and acquires the medical image 40 by performing imaging under the imaging conditions corresponding to the checked imaging order. The medical image 40 obtained by imaging is stored in the medical image DB server 14 together with the patient ID of the patient 36 to be imaged (subject), the imaging part or direction, the imaging conditions, imaging date and time, the type of the modality 12 used in the imaging, the ID of the user 52 in charge of imaging, and the like.

In a case where an interpretation request is included in the imaging order, the RIS transmits, to the interpretation terminal 18, the order of interpretation of the medical image 40 obtained by imaging. The interpretation doctor 44 checks the order through the interpretation terminal 18, and creates the interpretation report 50 according to the checked order. The created interpretation report 50 is transmitted to the interpretation DB server 20 and stored therein. The treatment department doctor (or the clinician) 42 accesses the interpretation DB server 20 from the clinical terminal 16, and determines the treatment policy of the patient 36 or performs treatment with reference to the interpretation report 50.

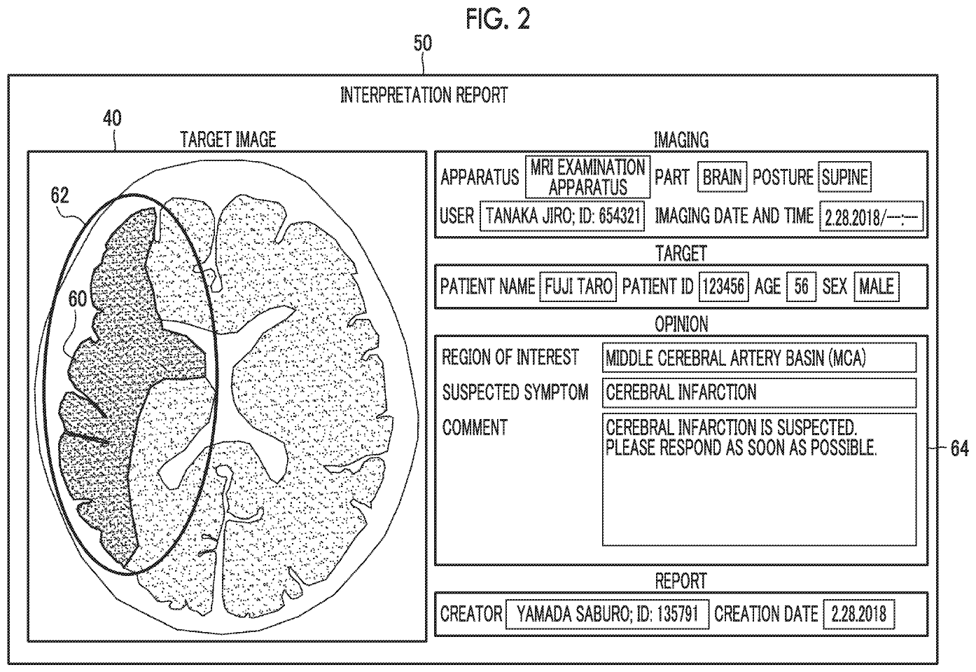

As shown in FIG. 2, in the interpretation report 50, the medical image 40 to be interpreted and various kinds of information regarding the medical image 40 are associated with each other. Various kinds of information regarding the medical image 40 include the type of the modality 12 used for imaging, the name of the patient 36 to be imaged, the patient ID, the imaging part or direction, imaging date and time, the name of an interpretation report creator, a creation date, and the like. In addition, a finding 64 on the medical image is also included in the various kinds of information regarding the medical image 40. In the present embodiment, an example is shown in which a region to which attention is to be paid (region of interest) 60 is surrounded by an indicator 62 in the medical image 40 and the opinion of the interpretation doctor 44 regarding the region of interest 60 is described as the finding 64.

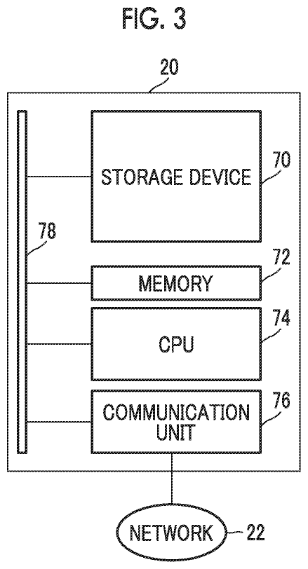

In FIG. 3, the interpretation DB server 20 is a known computer, and comprises a storage device 70 (storage unit), a memory 72, a central processing unit (CPU) 74, and a communication unit 76. These are connected to each other through a data bus 78. The storage device 70 is a hard disk drive, which is built into a computer that forms the interpretation DB server 20 or the like or which is connected to the computer through a cable or a network, or a disk array formed by connecting a plurality of hard disk drives. The memory 72 is a work memory for the CPU 74 to execute processing. The CPU 74 performs overall control of each unit of the computer by loading a program stored in the storage device 70 to the memory 72, thereby executing the processing according to the program. The communication unit 76 is a network interface to perform transmission control of various kinds of information through the network 22.

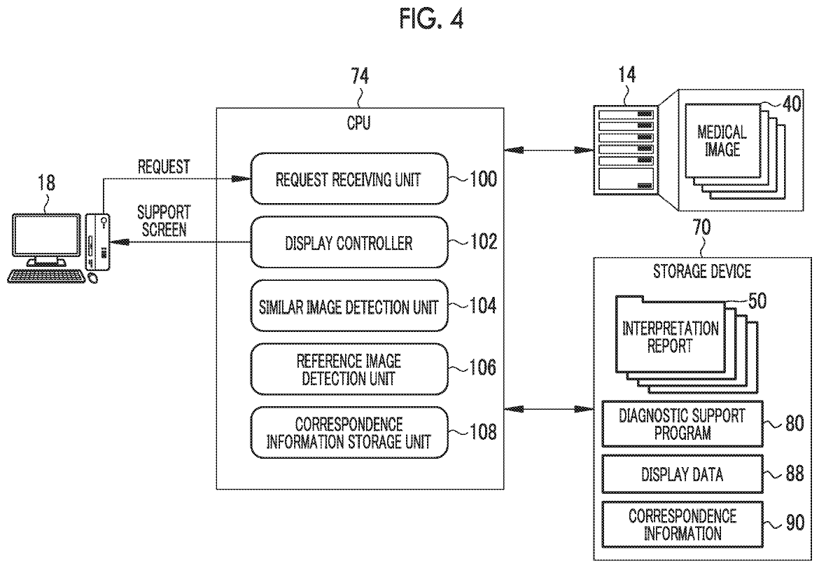

As shown in FIG. 4, in addition to the interpretation report 50 described above, a control program such as an operating system, various application programs, and various kinds of data attached to these programs are stored in the storage device 70. Specifically, a diagnostic support program 80 is stored as an application program, and display data 88 for displaying diagnostic support screens 82, 84 and the like and correspondence information 90 to be described later are stored as data attached to the diagnostic support program 80.

The diagnostic support program 80 is an application program for causing a computer configuring the interpretation DB server 20 to function as a diagnostic support apparatus for supporting diagnosis (in the present embodiment, interpretation (creation of an interpretation report)). The diagnostic support program 80 is activated by accessing the interpretation DB server 20 from the interpretation terminal 18 and inputting an operation request. In a case where the diagnostic support program 80 is activated, the CPU 74 of the interpretation DB server 20 cooperates with the memory 72 and the like to function as a request receiving unit 100, a display controller 102, a similar image detection unit 104, a reference image detection unit 106, and a correspondence information storage unit 108.

The request receiving unit 100 receives various requests input from the interpretation terminal 18 through the diagnostic support screens 82 and 84 or the like. With the operation of the diagnostic support program 80, the display controller 102 displays a selection screen (not shown) for selecting the medical image 40 (hereinafter, referred to as a target image 40a), which is a current diagnostic target (target for whom the interpretation report 50 is to be created), from the medical images 40 stored in the medical image DB server 14 (refer to FIG. 1) on the display of the interpretation terminal 18. On the selection screen, the target image 40a can be selected, for example, by designating a patient and/or an interpretation request (order for interpretation) or by designating one of a list of medical images 40 stored in the medical image DB server 14.

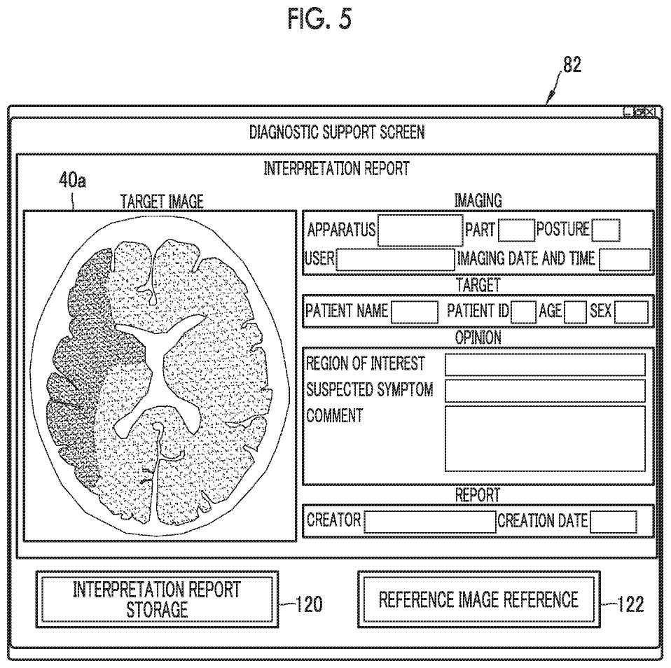

As shown in FIG. 5, in a case where the target image 40a is selected, the display controller 102 displays the diagnostic support screen 82 for supporting the creation of an interpretation report of the selected target image 40a on the display of the interpretation terminal 18. Compared with the interpretation report 50 shown in FIG. 2, on the diagnostic support screen 82, there is no processing (setting of the region of interest 60, the indicator 62, or the like) on the medical image 40 (target image 40a), and items (finding and the like) of various kinds of information regarding the medical image 40 (target image 40a) are blank. The interpretation doctor 44 completes the interpretation report by inputting information in a blank portion and setting the region of interest 60, the indicator 62, and the like as necessary, and selects an interpretation report saving tag 120 by clicking or the like. As a result, the new interpretation report 50 is created and saved (stored) in the storage device 70.

In addition, a reference image reference tag 122 is provided on the diagnostic support screen 82. The reference image reference tag 122 is selected in the case of referring to the past diagnosis in the process of creating the interpretation report 50 according to the diagnostic support screen 82. Then, in a case where the reference image reference tag 122 is selected, the similar image detection unit 104 and the reference image detection unit 106 operate.

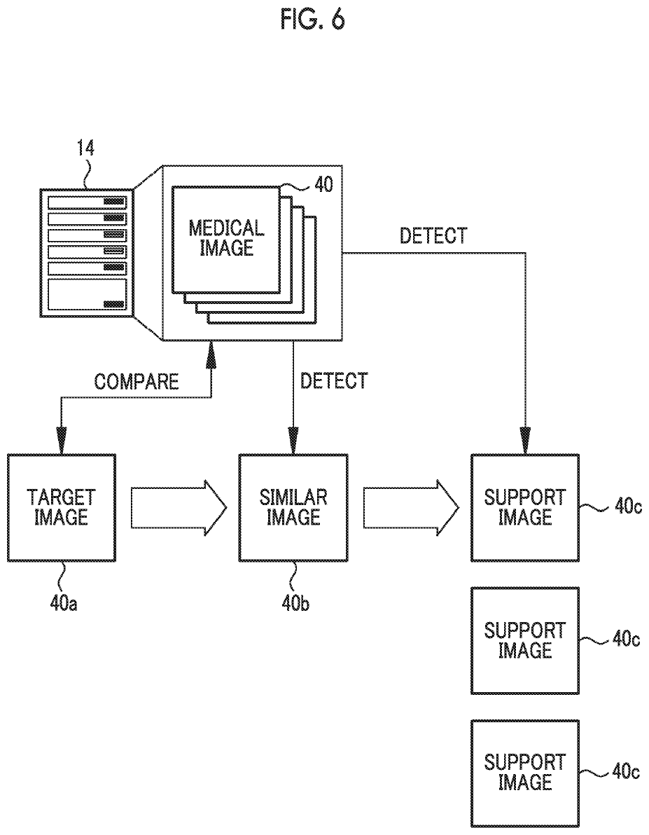

As shown in FIG. 6, the similar image detection unit 104 compares the target image 40a, which is a current diagnostic target, with each of the medical images 40 (excluding the target image 40a) stored in the medical image DB server 14, and calculates the degree of similarity with the target image 40a. Then, the medical image 40 having the highest degree of similarity (hereinafter, referred to as a similar image 40b) is detected from the medical image DB server 14. Although the present embodiment is described by way of an example in which one medical image 40 is detected as the similar image 40b, two or more similar images 40b may be detected. In the case of detecting a plurality of similar images 40b, the detection may be performed in the descending order of the degree of similarity.

The reference image detection unit 106 detects the medical image 40 for supporting the diagnosis of the target image 40a that is a current diagnostic target (hereinafter, referred to as a support image 40c) from the medical image DB server 14. Here, the support image 40c is the medical image 40 referred to in a case where the similar image 40b was a diagnostic target in the past diagnosis, and is an image corresponding to the reference image of the invention. The storage device 70 (refer to FIG. 4) stores the correspondence information 90 indicating the correspondence relationship between each medical image 40 for which the interpretation report 50 has already been created (that is, the medical image 40 for which diagnosis has already been completed in the past), among the medical images 40 stored in the medical image DB server 14, and the medical image 40 referred to in a case where each medical image 40 is diagnosed. The reference image detection unit 106 accesses the storage device 70 to refer to the correspondence information 90, and detects, as the support image 40c, the medical image 40 referred to in a case where the similar image 40b was a diagnostic target in the past diagnosis.

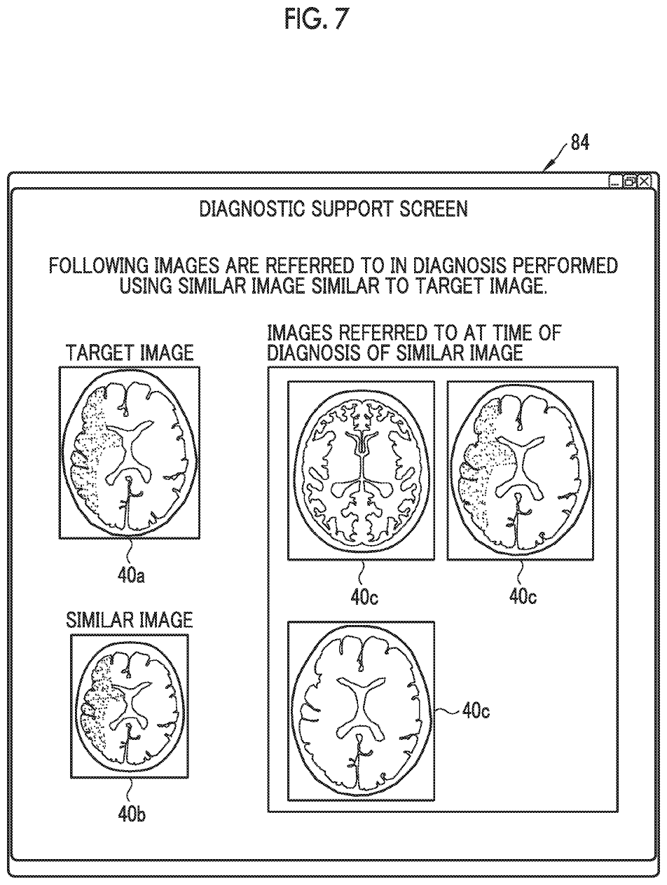

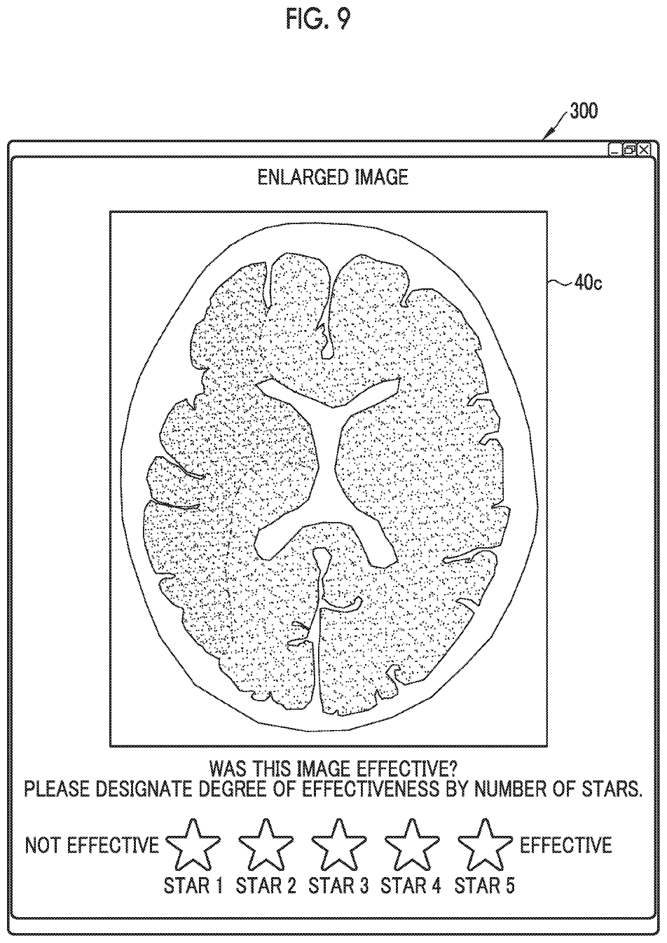

As shown in FIG. 7, in a case where the similar image 40b and the support image 40c are detected, the display controller 102 creates the diagnostic support screen 84 including the similar image 40b and the support image 40c and displays the diagnostic support screen 84 on the display of the interpretation terminal 18. On the diagnostic support screen 84, the support images 40c are displayed so as to be listed as thumbnail images subjected to reduction processing. In a case where one of the thumbnail images (support images 40c) is selected, the selected support image 40c is enlarged and displayed (for example, an enlarged screen 300 shown in FIG. 9 is displayed). The interpretation doctor 44 can diagnose the target image 40a with reference to the support image 40c that is enlarged and displayed as described above.

In a case where the support image 40c is referred to (enlarged and displayed) as described above, the correspondence information storage unit 108 (refer to FIG. 4) operates to update the correspondence information 90 (refer to FIG. 4). Specifically, the correspondence information 90 is updated by adding the content indicating that the referred support image 40c is an image referred to in the case of diagnosing the target image 40a.

As described above, in the present embodiment, in the case of making a diagnosis using the target image 40a, an image referred to at the time of diagnosis of the similar image 40b similar to the target image 40a is displayed as the support image 40c. Therefore, by referring to the support image 40c, it is possible to perform a diagnosis by taking the past cases into consideration more reliably than in a case where the interpretation doctor 44 examines past cases independently. In addition, since cases (past cases) to be referred to differ depending on the interpretation doctor 44, it is also possible to prevent a problem, such as a variation in diagnosis by the interpretation doctor 44.

The invention is not limited to the embodiment described above, and the specific configuration can be appropriately changed. For example, in the embodiment described above, in order to support the diagnosis (interpretation and creation of the interpretation report 50) performed by the interpretation doctor 44, an example in which the support image 40c is displayed on the interpretation terminal 18 has been described. However, in order to support the diagnosis (specification of a disease name, creation of an electronic medical record, and the like) performed by the treatment department doctor 42, the support image 40c may be displayed on the clinical terminal 16.

As described above, in the case of displaying the support image 40c on the clinical terminal 16, the support image 40c is preferably stored in, for example, an electronic medical record of the patient 36 as patient information regarding the patient 36 of the subject of the target image 40a. In this case, with the activation of the diagnostic support program 80, the CPU 74 of the interpretation DB server 20 may be made to function as a support image storage unit (reference image storage unit) that stores the support image 40c as patient information of the patient 36 of the subject of the target image 40a. Similarly, patient information (electronic medical record or the like of the patient) regarding the patient of the subject of the support image 40c may be stored as patient information regarding the patient 36 of the subject of the target image 40a. In this case, with the activation of the diagnostic support program 80, the CPU 74 of the interpretation DB server 20 may be made to function as a patient information storage unit that stores patient information regarding the patient of the subject of the support image 40c as patient information of the patient 36 of the subject of the target image 40a. In this manner, it is possible to determine the treatment policy on the patient 36 of the subject of the target image 40a with reference to the treatment performed on the patient of the subject of the support image 40c, the progress thereof, and the like.

Although the present embodiment has been described by way of an example of detecting the similar image 40b similar to the entire target image 40a, the invention is not limited thereto. In the target image 40a, for example, the region of interest 60 or the like may be set (a region may be designated) by the indicator 62 or the like (refer to FIG. 2). In such a case, for a portion in which the region of interest 60 (region surrounded by the indicator 62) is set (designated region) in the target image 40a, the similar image 40b including a portion similar to such a portion (region) may be detected as a similar image.

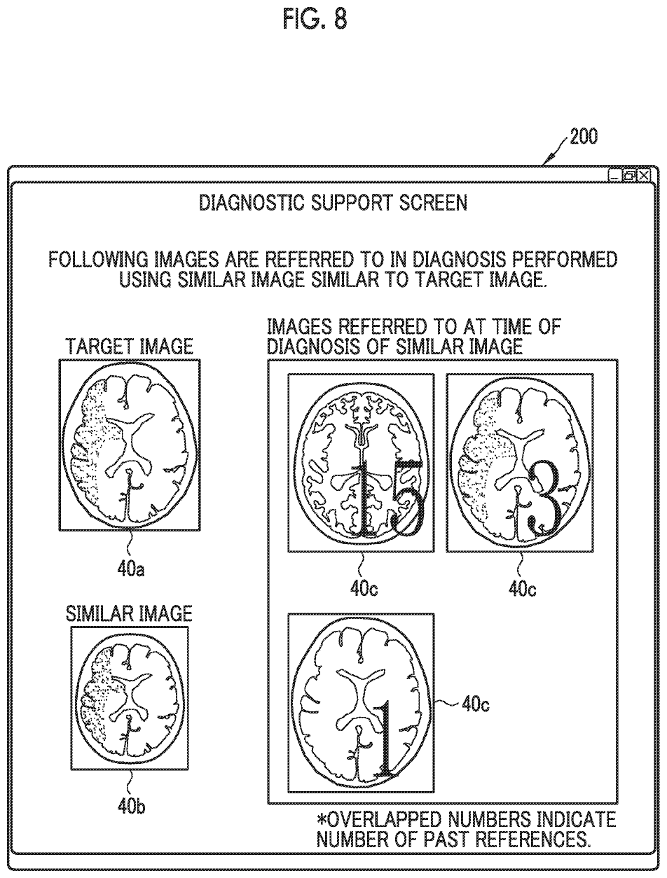

Although the present embodiment has been described by way of an example in which images referred to in the past diagnosis performed using the similar image 40b are uniformly displayed as the support images 40c, the invention is not limited thereto. For example, the number of times by which the support image 40c has been referred to (enlarged and displayed) may be stored so as to be associated with the support image 40c or the correspondence information 90 (refer to FIG. 4), and the display mode of the support image 40c may be changed based on the number of times referred to in the past, for example, by displaying the images in order from an image with the highest number of times of reference or displaying only the images with the number of times of reference equal to or greater than a threshold value. Alternatively, as in a diagnostic support screen 200 shown in FIG. 8, the number of times of reference in the past may be displayed so as to be associated with the support image 40c, for example, so as to overlap the support image 40c.

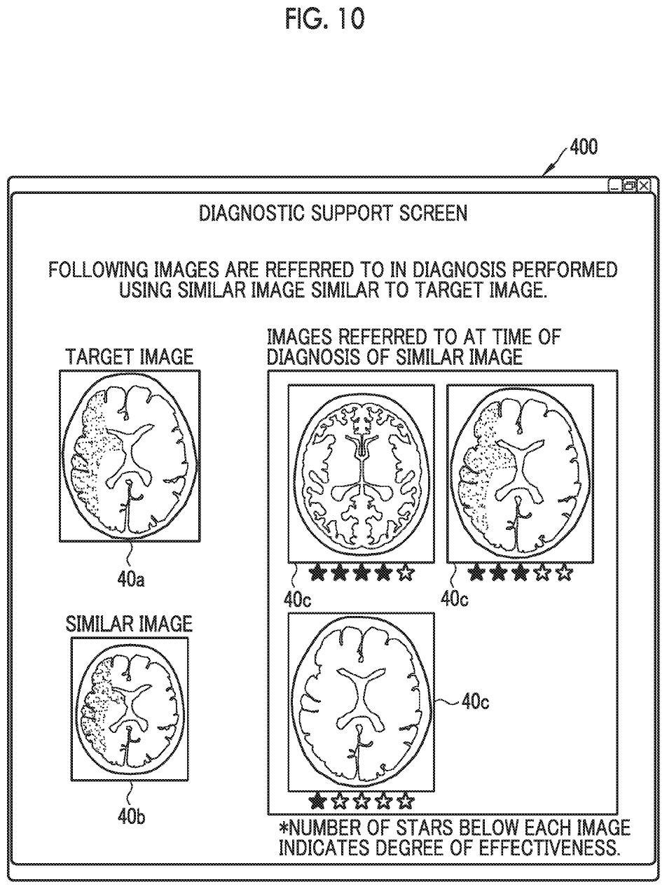

Even the image referred to in the past diagnosis has various influences on the diagnosis. Accordingly, such information (the amount of influence on the past diagnosis) may be stored as the degree of effectiveness so as to be associated with the support image 40c or the correspondence information 90 (refer to FIG. 4), and the display mode of the support image 40c may be changed based on the degree of effectiveness, for example, by displaying the images in order from an image with the highest degree of effectiveness or displaying only the images with the degree of effectiveness equal to or greater than a threshold value. In this case, as in the enlarged screen 300 shown in FIG. 9, the user (the interpretation doctor 44 or the treatment department doctor 42) may designate the degree of effectiveness in a case where the support image 40c is referred to (enlarged and displayed). Needless to say, as in a diagnostic support screen 400 shown in FIG. 10, the degree of effectiveness may be displayed so as to be associated with the support image 40c.

In the case of making the user who has referred to the support image 40c designate the degree of effectiveness as shown in FIG. 9, a comment input field may be provided on the enlarged screen 300 so that a comment, such as a reason why the degree of effectiveness has been designated (why the value (the degree of effectiveness) has been designated), is input to the comment input field, and the input comment may be displayed so as to be associated with the support image 40c on the diagnostic support screen 400 shown in FIG. 10, for example. For the support image 40c having a comment thereon, for example, an icon indicating that there is a comment may be displayed on the enlarged screen 300 (refer to FIG. 9) and/or the diagnostic support screen (refer to FIG. 10), so that the comment is displayed in a case where the icon is selected by a clicking operation. Needless to say, the case where a comment is input is not limited to the example of displaying the comment input field from the beginning, and an icon for displaying the comment input field or the like may be displayed, and the comment input field may be displayed so that it is possible to input a comment in a case where the icon is selected. By allowing the input or viewing of a comment as described above, there is no doubt or misunderstanding as to why an interpretation doctor who referred to the support image 40c in the past diagnosis made such an evaluation. As a result, current diagnosis can be performed more efficiently.

In the examples shown in FIGS. 9 and 10, the degree of effectiveness is evaluated as one of five steps. However, the degree of effectiveness may be evaluated as one of four or less steps, or may be evaluated as one of five or more steps. For example, in FIG. 10, in a case where one support image 40c is referred to multiple times in the past diagnosis, that is, in a case where the evaluation of the degree of effectiveness is input multiple times, it is preferable to display the average of the degree of effectiveness input in the past diagnosis so as to be associated with the support image 40c. In the case of displaying the average of the degree of effectiveness as described above, the average of the degree of effectiveness may not belong to any of the five steps even though the degree of effectiveness has been input as one of the five steps, for example. Specifically, in a case where one support image 40c was referred to twice in the past diagnosis and “3” was input as the degree of effectiveness in the first reference and “4” was input as the degree of effectiveness in the second reference, the average of the degree of effectiveness is “3.5”. In such a case, the average of the degree of effectiveness may be displayed so as to be subdivided (for example, rounded off in units of 0.5) within a predetermined effective range. Specifically, in a case where the average of the degree of effectiveness is equal to or greater than “3.25” and less than “3.75”, the average of the degree of effectiveness may be set to “3.5” and displayed so as to be associated with the support image 40c. As a display showing the degree of effectiveness “3.5” on the diagnostic support screen 400 shown in FIG. 10, three marks of the five star-shaped marks may be entirely filled, a half (for example, a left half) of one mark may be filled, and the remaining one mark may be unfilled.

Although an example of displaying the support image 40c has been described, an image referred to in the past diagnosis using the support image 40c (a secondary reference image of the invention; hereinafter, referred to as a secondary support image) may be displayed in addition to the support image 40c. In this case, the secondary support image may be treated in the same manner as the support image 40c and may be displayed as a type of the support image 40c, or the secondary support image may be displayed as an image referred to at the time of diagnosis of the enlarged support image 40c only in a case where the support image 40c is referred to (enlarged and displayed).

In addition, although an example in which the similar image 40b is displayed so as to be distinguished from the support image 40c (displayed in a separate field) has been described in the above embodiment, the similar image 40b may not be displayed (the field for displaying the similar image 40b may be eliminated). Alternatively, the similar image 40b may be displayed in the same column as the support image 40c (in the same field as the support image 40c) as a type of the support image 40c.

### EXPLANATION OF REFERENCES

10: medical information system

12: modality

14: medical image DB server

16: clinical terminal

18: interpretation terminal

20: interpretation DB server (diagnostic support apparatus)

22: network

30: CT examination apparatus

32: MRI examination apparatus

34: X-ray examination apparatus

36: patient

40: medical image

40a: target image

40b: similar image

40c: support image (reference image)

42: treatment department doctor (clinician)

44: interpretation doctor

50: interpretation report

52: user

60: region of interest

62: indicator

64: opinion

70: storage device

72: memory

74: CPU

76: communication unit

78: data bus

80: diagnostic support program

82, 84, 200, 400: diagnostic support screen

88: display data

90: correspondence information

100: request receiving unit

102: display controller

104: similar image detection unit

106: reference image detection unit

108: correspondence information storage unit

120: interpretation report storage tag

122: reference image reference tag

300: enlarged screen

What is claimed is:

1. A diagnostic support apparatus for supporting a diagnosis performed using a medical image, comprising:
a correspondence information storage unit that stores correspondence information indicating a correspondence relationship between a medical image, which is a diagnostic target in a past diagnosis, and a medical image referred to in the past diagnosis;
a similar image detection unit that sets a medical image, which is a current diagnostic target, as a target image and detects a similar image, which is similar to the target image, from medical images that are diagnostic targets in the past diagnosis;
a reference image detection unit that detects a reference image, which is a medical image referred to in diagnosing the similar image, using the correspondence information; and
a display controller that displays the reference image on a display unit.

2. The diagnostic support apparatus according to claim 1,
wherein a plurality of the reference images are displayed as a list.

3. The diagnostic support apparatus according to claim 1,
wherein the reference image is displayed on the display unit as a thumbnail image subjected to reduction processing.

4. The diagnostic support apparatus according to claim 1, further comprising:
a reference image storage unit that stores the reference image so as to be associated with patient information regarding a patient of a subject of the target image.

5. The diagnostic support apparatus according to claim 1, further comprising:
a patient information storage unit that stores patient information regarding a patient of a subject of the reference image so as to be associated with patient information regarding a patient of a subject of the target image.

6. The diagnostic support apparatus according to claim 1,
wherein the similar image detection unit detects an image, which includes a region similar to a designated partial region of the target image, as the similar image.

7. The diagnostic support apparatus according to claim 1,
wherein the correspondence information includes a degree of effectiveness indicating a degree of influence on the past diagnosis, and
the display controller determines a display mode of the reference image using the degree of effectiveness.

8. The diagnostic support apparatus according to claim 7,
wherein the display controller displays the degree of effectiveness so as to be associated with the reference image.

9. The diagnostic support apparatus according to claim 7,
wherein the display controller determines a display order of the reference image using the degree of effectiveness.

10. The diagnostic support apparatus according to claim 2,
wherein the correspondence information includes the number of references in the past diagnosis, and
the display controller determines a display mode of the reference image using the number of references.

11. The diagnostic support apparatus according to claim 10,
wherein the display controller displays the number of references so as to be associated with the reference image.

12. The diagnostic support apparatus according to claim 10,
wherein the display controller determines a display order of the reference image using the number of references.

13. The diagnostic support apparatus according to claim 1,
wherein the reference image detection unit detects an image referred to in a past diagnosis in which the reference image is a diagnostic target, as a secondary reference image, using the correspondence information, and
the display controller displays the secondary reference image on the display unit.

14. The diagnostic support apparatus according to claim 1,
wherein the display controller displays the similar image on the display unit as the reference image.
